(19) United States
(12) Patent Application Publication
(10) Pub. No.: US 2024/0141398 A1
(43) Pub. Date: May 2, 2024

(54) A METHOD FOR PREPARATION OF alpha-GLUCAN

(71) Applicants: Jianhua ZHU, Edmonton (CA); Xiaolin Li, Huannge Town, Nansha Districtistrict, (CN)

(72) Inventors: Jianhua ZHU, Edmonton (CA); Xiaolin LI, Guangdong (CN); Jinghua ZENG, Guangdong (CN)

(21) Appl. No.: 18/279,596

(22) PCT Filed: Apr. 8, 2022
(86) PCT No.: PCT/CA2022/050554

§ 371 Date: Aug. 30, 2023

(30) Foreign Application Priority Data
Mar. 1, 2021 (CN) 202110224832.1

(51) Int. Cl. C12P 19/08; A23L 33/135; A23L 33/21; C12N 1/20; C12N 9/46; C12P 19/14; C12R 1/00

(52) U.S. Cl. CPC C12P 19/08; A23L 33/135; A23L 33/21; C12N 1/205; C12N 9/2454; C12P 19/14; C12N 2500/12; C12N 2500/34; C12N 2500/74; C12N 2523/00; C12R 2001/00

(57) ABSTRACT

A production method of low molecular weight α-glucan includes: inoculating an activated Leuconostoc mesenteroides in a 5 L fermentor at a 10% inoculum. Fermentation broth is placed in the fermentor at an initial pH of 6.8-7.0, temperature of 25° C. to 28° C., stirring speed at 120 r/min, and fermented for 20-40 hours. Dextranase is added after 5-30 hours of fermentation at a dosage of 1/10,000 to 5/10,000 by volume. The molecular weight of α-glucan is controlled within 10000D by the amount of enzyme added, and the total fermentation process is about 20-40 hours. After the reaction is terminated, the fermentation liquid is concentrated and dried to prepare dietary fiber products with a molecular weight of 500-5000D. The viscosity of the fermentation liquid and concentration of α-glucan in the fermentation liquid may be reduced to promote the forward reaction, accelerate the sucrose conversion rate and increase the product yield.

### TECHNICAL FIELD

This relates to biological fermentation and dietary fiber, and in particular, a method of producing low molecular weight α-glucan.

### BACKGROUND

With the prevalence of human obesity and the three common symptoms of high blood cholesterol, high blood pressure, and high blood sugar, functional carbohydrates with low Glycemic Index and low calorie content have become the developing trend of healthy food in the 21st century to comply with the development of modern food science and technology and meet the demand of the health of consumers. Functional polysaccharides have increasingly become the focus of attention.

Glucan is an extracellular polysaccharide produced by lactic acid bacteria. It is an oligo-/poly-saccharide with a special structure secreted outside the cell wall during the metabolism and growth of lactic acid bacteria. It is a functional polysaccharide with good stability at low pH and high heat, has good anti-digestibility properties, has a low Glycemic Index, low insulin index, and beneficial prebiotics properties such as low calorie, anti-cavity, and can promote the growth of intestinal probiotics and maintain the microbial balance of gut microbiota.

The oligo-/poly-saccharides are produced by fermentation at a low cost and are not limited by seasonal and regional environmental conditions. Fermentation methods are of great significance to the industrial production of oligo/polysaccharides. Leuconostoc mesenteroides is one of the 42 microorganisms safe for direct consumption listed by the FDA and AAFCO in 1998 and listed in the List of Culture Species for Use in Foodstuff issued by the Ministry of Health of the People's Republic of China in 2012. During the metabolism and growth of Leuconostoc mesenteroides, dextransucrase is produced out of the cell, with the inexpensive and environmentally friendly sucrose used as the donor, the fructosyl and glucosyl moieties of sucrose will polymerize into α-glucan with the main chain linked by α-(1, 6) glycosidic bonds and branched by α-(1, 2), α-(1, 3) α-(1, 4) glycosidic bonds. The molecular weight is distributed from tens of thousands to millions, which are sticky.

Dextranase can cut off the glycosidic bond of α-glucan to reduce the molecular weight of α-glucan to produce low molecular weight α-glucan, which reduces viscosity while increasing usability. This low molecular weight α-glucan is a polysaccharide because dextranase is only specific to α-(1,6) glycosidic bonds and will not cleave other glycosidic bonds. The low molecular weight α-glucan after enzymatic hydrolysis is still mixed with different branched structures formed by α-(1,2), α-(1,3), and α-(1,4) glycosidic bonds. These special molecular structures make it have digestive tolerance, immune and anti-cancer properties, prevent hypertension and arteriosclerosis, and help weight loss. The molecular weight of α-glucan is controlled below 5000 D. The product is easily soluble in cold water, making it a high-quality new diet fiber food raw material, which can be used as a low-glycemic sweetener and prebiotics.

Under normal conditions, the fermentation of Leuconostoc mesenteroides to produce α-glucan can increase the viscosity of the fermentation liquid and gradually reduce the reaction rate. Dextranase enzymatic reaction and fermentation biosynthesis are almost simultaneously processed in the fermentation tank. It can be carried out to reduce the viscosity of the fermentation liquid and at the same time reduce the concentration of the polymer α-glucan in the fermentation liquid so that the reaction can continue to proceed forward, accelerate the sucrose conversion rate, and increase the product yield.

Fructose, a by-product of the reaction, is remained in the fermentation liquid. In the post-treatment of the fermentation liquid, fructose is separated by chromatographic separation and further utilized to produce fructan.

### SUMMARY

According to an aspect, there is provided a preparation method for α-glucan dietary fiber that uses sucrose metabolism and transformation of Leuconostoc mesenteroides to generate α-glucan; at the same time, dextranase is added at an appropriate time in the reaction process to form and degrade α-glucan. An α-glucan product with high molecular weight and high viscosity is degraded by the enzymatic reaction so that the reaction progresses smoothly forward. The utilization rate of the raw material substrates and the dietary fiber properties of the products may be further improved by optimizing Leuconostoc mesenteroides and the conditions of fermentation and enzymatic reactions.

According to an aspect, the method of preparing α-glucan may include the following:
(1) A Leuconostoc mesenteroides strain may use sucrose, the main component of white sugar, raw sugarcane sugar, and sugarcane juice, as the primary nutrient for growth and metabolism, to extracellularly produce dextransucrase, which cleaves sucrose into fructosyl and glucosyl moieties, which may polymerize again to form α-glucan, mainly linked by α-(1,6) glycosidic bond and branched by α-(1,2), α-(1,3), and α-(1,4) glycosidic bonds.
(2) When the above polymerization reaction proceeds to a certain extent, dextranase is added to cut the glycosidic bonds of α-glucan to reduce the molecular weight of α-glucan and produce low molecular weight α-glucan dietary fiber.
(3) The molecular weight of α-glucan is controlled in the reaction solution by controlling the adding time, adding amount, and reaction time of dextranase.
(4) After the initial treatment of the filtration and ion exchange process, the reaction solution is further separated by chromatographic separation to remove the small molecules α-glucan less than 500 D and the large molecules α-glucan higher than 5000 D. More than 95% of the final product is a α-glucan dietary fiber with molecular weight within 500-5000 D, which can meet special functional requirements.

The optimal dextransucrase-producing strains in Step (1) may be Leuconostoc mesenteroides CICC-23614, Leuconostoc mesenteroides LM-1226, Leuconostoc mesenteroides LM-0326, Leuconostoc mesenteroides Lm-31208, which may be inoculated into a 5 L fermentation tank with 10% seed culture after activation.

Further, considering the cost of production and entirely reverting the natural growth attributes of the strains and the overall requirements for nutrition, the carbon sources of the fermentation medium in Step (1) may be white sugar, raw sugarcane sugar, sugarcane juice, and the dosage is 10%, 15%, 18%, 20%, 25%, 30%, and the concentrations are all based on sucrose; the nitrogen sources are tryptone, yeast powder, and their 1:1 mixture, at a dosage of 0.3%.

Further, the initial pH of the fermentation broth described in Step (1) may be 6.8-7.0, which may be adjusted with sodium carbonate; the temperature of the fermentation broth described in Step (1) may be 28° C.

Further, the time of adding dextranase in Steps (2) and (3) may be after 5-30 hours, and the amount may be 1/10,000 of the total fermentation broth.

Further, the total reaction time described in Steps (1), (2), and (3) may be 20-40 hours, and the molecular weight of the product in the reaction solution may be controlled such that at least 95% is within 10000 D.

Further, the chromatographic separation in Step (4) may be used to control more than 90% of the final product within 500-5000 D.

According to an aspect, the method may utilize the sucrose metabolism and transformation of Leuconostoc mesenteroides to generate α-glucan; and dextranase may be added at an appropriate time in the reaction process to make the reaction and degradation of α-glucan carry out at the same time. Through the optimization of Leuconostoc mesenteroides and the optimization of the fermentation and enzymatic hydrolysis reaction conditions, the sucrose conversion rate in the fermentation broth may reach more than 90%, and the proportion of α-(1,6) glycosidic bond in the molecular structure of the product may reached more than 80%. Finally, the α-glucan dietary fiber product with a target molecular weight of 500-5000 D may be obtained through post-processing such as decolorization filtration, ion exchange, chromatographic separation, etc. The sum of the anti-digestibility index and slow-digestibility index may reached more than 90%. This method of producing new dietary fiber food raw materials through the fermentation of sucrose-containing raw materials by Lactobacillus spp., combined with enzymatic biological engineering new technology, may have a wide range of raw materials sources and low prices. The method may be used to in a production process that is relatively easy to implement, to produce a stable and controllable product quality that is relatively safe and reliable. The lactic acid bacteria biological fermentation method may produce functional dietary fiber.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a molecular weight distribution diagram of the product obtained in Example 14 described herein.

FIG. 2 is a molecular weight distribution diagram of the product obtained in Example 15 described herein.

FIG. 3 is a molecular weight distribution diagram of the product obtained in Example 16 described herein.

FIG. 4 is a molecular weight distribution diagram of the product obtained in Example 17 described herein.

FIG. 5 is a molecular weight distribution diagram of the product obtained in Example 18 described herein.

FIG. 6 is a molecular weight distribution diagram of the product obtained in Example 19 described herein.

FIG. 7 is a characteristic diagram of glycosidic bonds of the product obtained in Example 16 described herein.

FIG. 8 is a characteristic diagram of glycosidic bonds of the product obtained in Example 17 described herein.

### DETAILED DESCRIPTION OF PREFERRED EMBODIMENTS

There will now be described examples of processes used to prepare α-glucan dietary fiber.

### Example 1

(1) The lyophilized strain of Leuconostoc mesenteroides CICC-23614 was transferred to the 5 L liquid fermentation tank (liquid fermentation medium I) with 10% inoculum after two slant transfers and one liquid seed culture activation. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 2

(1) The lyophilized strain of Leuconostoc mesenteroides LM-0326 was transferred to the 5 L liquid fermentation tank (liquid fermentation medium I) with 10% inoculum after two slant transfers and one liquid seed culture activation. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 3

(1) The lyophilized strain of Leuconostoc mesenteroides Lm-31208 was transferred to the 5 L liquid fermentation tank (liquid fermentation medium I) with 10% inoculum after two slant transfers and one liquid seed culture activation. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 4

(1) The lyophilized strain of Leuconostoc mesenteroides LM-1226 was transferred to the 5 L liquid fermentation tank (liquid fermentation medium I) with 10% inoculum after two slant transfers and one liquid seed culture activation. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 5

(1) The activated seed culture of Leuconostoc mesenteroides CICC-23614 was transferred to the 5 L liquid fermentation tank (liquid fermentation medium II) with 10% inoculum. The white sugar was used as carbon source (the concentration of sucrose was 18%). Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 6

(1) The activated seed culture of Leuconostoc mesenteroides CICC-23614 was transferred to the 5 L liquid fermentation tank (liquid fermentation medium II) with 10% inoculum. The raw sugarcane sugar was used as a carbon source (the concentration of sucrose was 18%). The concentration of sucrose in raw sugarcane was 18%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 7

(1) The activated seed culture of Leuconostoc mesenteroides CICC-23614 was transferred to the 5 L liquid fermentation tank (liquid fermentation medium II) with 10% inoculum. The sugarcane juice was used as a carbon source (the concentration of sucrose was 18%). Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 8

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 10%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 9

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 15%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 10

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 18%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 11

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 20%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 12

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 25%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 13

(1) The activated seed culture of Leuconostoc mesenteroides CICC-23614 was transferred to the 5 L liquid fermentation tank (liquid fermentation medium II) with 10% inoculum. The concentration of sucrose in sugarcane juice was 30%. Fermentation was conducted at 28° C., sampling every 5 hours to detect sucrose residues and calculate the sucrose conversion rate;

(2) After 15 hours of fermentation, Dextranase Plus L was added at 1/10000 of the fermentation liquid to continue the reaction, and the amount was 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 14

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 5 hours of fermentation, Dextranase Plus L was added at 1/10000 of the total amount of fermentation liquid. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 15

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 10 hours of fermentation, Dextranase Plus L of 1/10000 of the total amount was added. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 16

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 15 hours of fermentation, Dextranase Plus L of 1/10000 of the total amount of fermentation liquid was added. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 17

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 20 hours of fermentation, Dextranase Plus L of 1/10000 of the total amount of fermentation liquid was added. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 18

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 25 hours of fermentation, Dextranase Plus L of 1/10000 of the total amount of fermentation liquid was added. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### Example 19

(1) The activated seed culture of Leuconostoc mesenteroides CICC-23614 was transferred to the 5 L liquid fermentation tank (liquid fermentation medium II) with 10% inoculum. The sugarcane juice was used as a carbon source with 20% sucrose. Fermentation was conducted at 28° C., sampling every 5 hours to detect sucrose residues and calculate the sucrose conversion rate;

(2) After 30 hours of fermentation, Dextranase Plus L of 1/10000 of the total amount of fermentation liquid was added. Samples were taken every 30 min to check the molecular weight distribution of the fermentation liquid;

(3) When more than 90% of the molecular weight of the liquid reaction product was controlled within 10000D, the reaction was terminated. The α-glucan syrup or powder with a molecular weight of 500-5000D was produced by filtration, ion exchange, Chromatographic separation, concentration, or spray drying.

### DISCUSSION

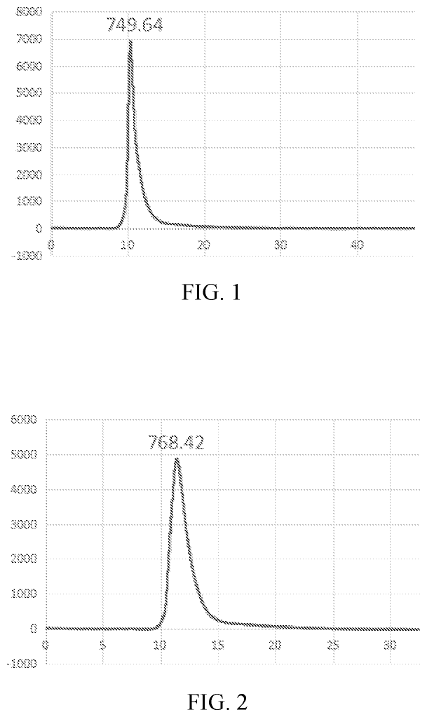

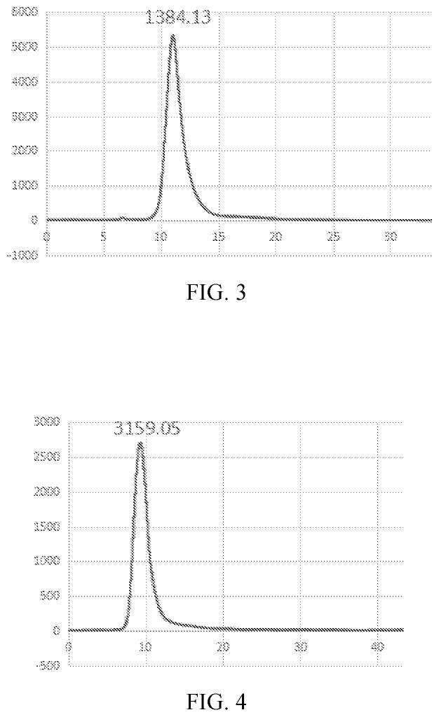

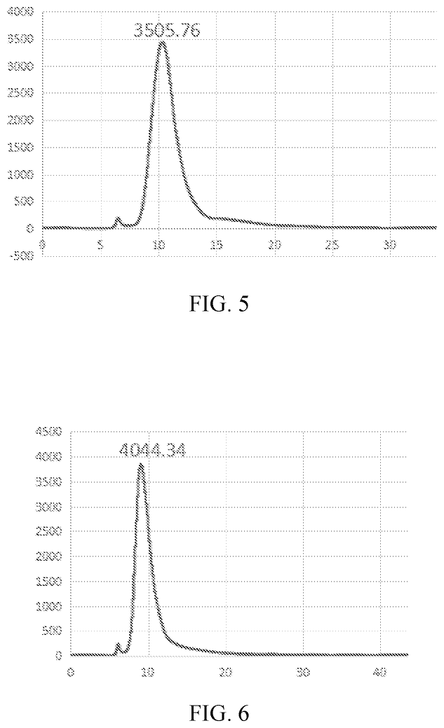

As can be seen from the data of the molecular weight distribution and the anti-digestibility in Tables 1, 2, 3, and 4 below and FIGS. 1, 2, 3, 4, 5, and 6, it may be beneficial to add Dextranase Plus L after 15-20 hours of fermentation, and the molecular weight distribution of the final product may be around 1000-3000D. It has been found that the product that results when Dextranase Plus L is added after 5 and 10 hours of fermentation has more small and medium molecular weight components and the post-treatment yield is low. The product that results when Dextranase Plus L is added after 25 and 30 hours of fermentation has higher molecular weight components. The post-treatment is more difficult, and the yield is low.

The final products of the above examples have a stable anti-digestibility between 82% and 90%. The whiteness of the products is over 70. The product does not easily absorb moisture.

The following analysis and measurement methods were used in the examples mentioned above:

1. Measurement Methods:
1.1 Calculation of sucrose conversion rate:
   1.1.1 Determination of sucrose residue: use high-performance liquid chromatography to quantitatively analyze and calculate the residue sucrose in the fermentation broth.
   Analysis conditions: detector: Shimadzu RID-10A; analytical column: shodex KS803; column temperature: 50° C.; flow rate: 1.0 mL/min; mobile phase: ultrapure water.
   1.1.2 Analysis and detection method in the above examples and chart examples.

Sucrose conversion rate (%)=(m initial sucrose−m sucrose residue)/m initial sucrose×100%
1.2 The digestibility method uses the enzyme kit produced by Megazyme, and the digestibility of the sample is analyzed according to the steps proposed by Englyst.
The specific steps are as follows:
   1.2.1 Enzyme Preparation
   Enzyme A: Porcine Pancreatic Alpha-Amylase (Model P7545)
   Take four 50 mL centrifuge tubes, add 3 g enzyme and 20 mL water each, add magnets and stir for 10 minutes (it needs to be fully dissolved, you can extend the time as needed), centrifuge for 10 minutes at 4300 r/min, and remove the supernatant from each tube Mix 13.5 mL of the solution to obtain 54 mL Enzyme A;
   Enzyme B: Amyloglucosidase (model A7095)
   Take 3.15 mL enzyme B and 3.6 mL water and mix, take 6 mL enzyme B from it;
   Enzymolysis solution: After mixing the 54 mL of enzyme A and 6 mL of enzyme B, add 4 mL of distilled water and refrigerate for later use.
   1.2.2 Buffer preparation
   1) Dissolve 13.6 g CH3COONa·3H2O in distilled water and dilute to 1 L;
   2) Adjust the pH of the solution in Step 1) to 5.2 with 0.1 mol/L acetic acid (the concentration of anhydrous acetic acid is 17.5 mol/L)
   3) Add 4 mL of 1 mol/LCaCl2 (molecular mass 111) per liter of buffer;
   4) If it needs to be stored for a longer time, add preservatives.
   1.2.3 Digestibility test
   1) Take 0.6 g sample (dry basis dry weight) in a 50 mL centrifuge tube, and set a blank control;
   2) Add 20 mL buffer, vortex to mix;
   3) Boil water bath for 30 minutes, keep shaking during the water bath, and then place it in a 37° C. water bath for cooling;
   4) After putting five glass beads in the centrifuge tube, place the centrifuge tube upright;
   5) Add 5 mL of mixed enzyme solution to the centrifuge tube, shake and mix, and place the centrifuge tube horizontally in a constant temperature water bath shaker (37° C., 160 stroke/min) and shake;
   6) After 20 minutes of reaction, take 0.25 mL of each reaction solution, add 10 mL of 66% ethanol, and centrifuge at 4300 r/min for 5 minutes. Take 0.1 ml of the supernatant, add 3 mL of GOPOD (kit) in a water bath at 50° C. for 20 minutes, and measure the absorbance at 510 nm. At the same time, take 0.1 mL of 1 mg/mL glucose standard solution, add 3 ml GOPOD (kit) in a water bath at 50° C. for 20 minutes, and test the absorbance at 510 nm. After reacting for 120 minutes, take 0.25 mL of the reaction solution, add 10 mL of 66% ethanol, and centrifuge at 4300 r/min for 5 minutes. Take 0.1 ml of the supernatant, add 3 mL of GOPOD (kit) in a water bath at 50° C. for 20 minutes, and measure the absorbance at 510 nm. At the same time, take 0.1 mL of 1 mg/mL glucose standard solution, add 3 mL GOPOD (kit), in a water bath at 50° C. for 20 minutes, and test the absorbance at 510 nm.

$$\% glucose = \frac{{A}_{t} \times {V}_{t} \times C \times D}{{A}_{s} \times {W}_{t}} \times 100$$

Aₜ=Absorbance of test solution
Vₜ=Total volume of test solution
C=1 Standard concentration (mg glucose/mL)=1
Aₛ=Absorbance of standard glucose
Wₜ=Sample weight
D=Dilution factor=40
RDS=(G20−FG)×0.9
SDS=(G120−G20)×0.9
RS=TS−(RDS+SDS)=TS−(G120×0.9)
RDS=Fast digestion content
SDS=Slow digestion starch content
RS=digestibility content FG=initial glucose content (calculated as 0)
TS=total starch
1.3. Whiteness detection
Experimental instrument: WSB-1 digital whiteness meter for whiteness detection (Hangzhou Qiwei Instrument Co., Ltd.)
1.4. Branching degree of α-glucan measurement (glycosidic bond features of the product), H-NMR measurement method:
H-NMR instrument name: 600M superconducting NMR spectrometer
Weigh a certain amount of sample and dissolve it in deuterium oxide, prepare it as a 4% sample, and then measure the H-NMR of the sample at room temperature.
1.5. Method of determination of molecular weight:
The molecular weight of the sample was determined by HPLC, the chromatographic column was Shodex KS-803 (8.0 mm×300 mm), and the detector was a refractive index detector. The sample was prepared as 2 mg/mL solution, filtered through a 0.45 μm membrane, ultrapure water was used as the mobile phase, the injection volume was 20 μL, the flow rate was 0.8 mL/min, the column temperature was 70° C., and the detector temperature was 50° C.

TABLE 1
Sucrose conversion rate of different
Leuconostoc mesenteroides strain numbers

| | Sucrose |
|---|---|
| Sucrose conversion rate of different Leuconostoc | conversion |
| mesenteroides strain numbers | rate (%) |
| Example 1 (Leuconostoc mesenteroides CICC-23614) | 95.2 |
| Example 2 (Leuconostoc mesenteroides LM-0326) | 87.5 |
| Example 3 (Leuconostoc mesenteroides Lm-31208) | 88.1 |
| Example 4 (Leuconostoc mesenteroides LM-1226) | 93.6 |

TABLE 2
Sucrose conversion rate of different raw materials

| Raw Material of carbon source | Sucrose conversion rate (%) |
|---|---|
| Example 5 (White sugar) | 90.5 |
| Example 6 (Raw sugarcane sugar) | 82.8 |
| Example 7 (Sugarcane juice syrup) | 94.2 |

TABLE 3
Sucrose conversion rate of different initial concentrations
of sugarcane juice used as a carbon source

| Sugarcane juice carbon source | Sucrose conversion |
|---|---|
| initial concentration (%) | rate (%) |
| Example 8 (Sugarcane juice used as a carbon | 80.2 |
| source at the initial concentration of 10%) | |
| Example 9 (Sugarcane juice used as a carbon | 86.9 |
| source at the initial concentration of 15%) | |
| Example 10 (Sugarcane juice used as a carbon | 93.5 |
| source at the initial concentration of 18%) | |
| Example 11 (Sugarcane juice used as a carbon | 94.9 |
| source at the initial concentration of 20%) | |
| Example 12 (Sugarcane juice used as a carbon | 95.1 |
| source at the initial concentration of 25%) | |
| Example 13 (Sugarcane juice used as a carbon | 95.6 |
| source at the initial concentration of 30%) | |

TABLE 4
Weight and content based on timing
of addition of Dextranase Plus L

| | | Anti- | Slow | |
|---|---|---|---|---|
| | Weight | digest- | digest- | Whiteness |
| | average | ibility | ibility | of the |
| Time of Dextranase Plus L | molecular | content | content | final |
| added to the reaction | weight | (%) | (%) | product |
| Example 14 (5 hours after | 749.64 | 82.95 | 4.36 | 72.7 |
| starting fermentation) | | | | |
| Example 15 (10 hours after | 768.42 | 84.94 | 8.82 | 72.9 |
| starting fermentation) | | | | |
| Example 16 (15 hours after | 1384.13 | 85.56 | 10.09 | 71.9 |
| starting fermentation) | | | | |
| Example 17 (20 hours after | 3159.05 | 85..53 | 12.30 | 72.2 |
| starting fermentation) | | | | |
| Example 18 (25 hours after | 3505.76 | 87.98 | 7.32 | 70.8 |
| starting fermentation) | | | | |
| Example 19 (30 hours after | 4044.34 | 91.35 | 4.63 | 68.5 |
| starting fermentation) | | | | |

TABLE 5
Glycosidic bond content based on addition of dextranase

| | α-(1,6) | α-(1,2) | α-(1,3) | α-(1,4) |
|---|---|---|---|---|
| Example | glycosidic | glycosidic | glycosidic | glycosidic |
| Glycosidic bond content | bond | bond | bond | bond |
| Example 16 (addition of | 80.13% | 1.60% | 0.66% | 8.13% |
| dextranase after 15 hours | | | | |
| of fermentation) | | | | |
| Example 17 (addition of | 82.15% | 3.34% | 2.98% | 0.27% |
| dextranase after 20 hours | | | | |
| of fermentation) | | | | |

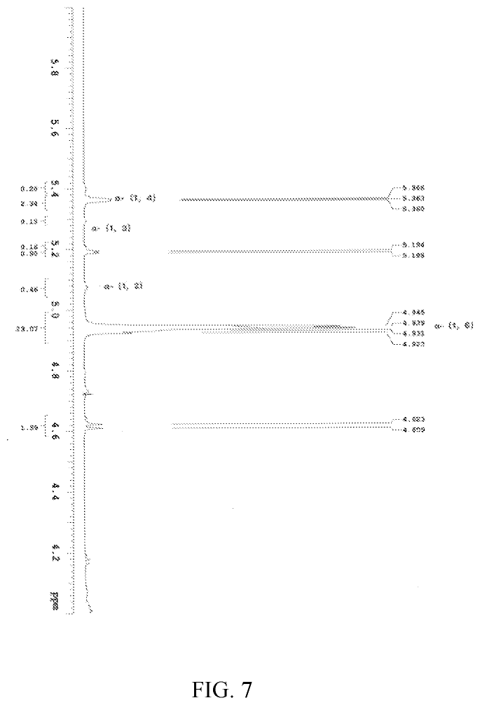

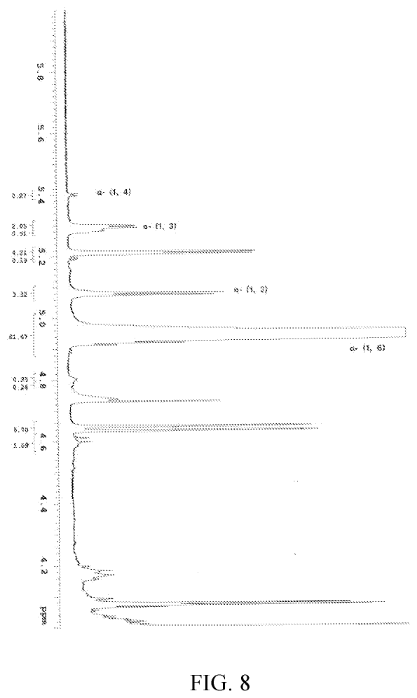

As can be seen, the molecules of α-glucan products mainly contain α-(1,6) glycosidic bonds, and there are a few α-(1,2), α-(1,3), and α-(1,4) glycosidic bonds on the branched chains. FIGS. 7 and 8 depict the molecular structure characteristics of α-glucan products.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings, but should be given the broadest interpretation consistent with the description as a whole.

What is claimed is:

1. A method for preparing α-glucan, comprising the steps of:
inoculating an activated culture of Leuconostoc mesenteroides in a 5 L fermenter with 10% inoculum, the fermenter comprising a fermentation broth that comprises:
a carbon source that provides a sucrose concentration of between 10-30% by weight;
a nitrogen source of 0.2-1.0%; and
an initial pH of between 6.8-7.0 and a temperature of 25° C. to 28° C. stirring the activated culture at a speed of 120 rpm;
after between 5-30 hours of fermentation, adding dextranase to the fermentation culture in an amount that is 1/10,0000 to 5/10,0000 of a total fermentation culture volume;
controlling a molecular weight of the α-glucan that is prepared in the fermentation culture to be within 10000D by monitoring the fermentation time and an amount of enzyme added;
after the reaction is terminated, the fermentation culture is decolorized and filtered through a plate and frame filter system and purified by ion exchange and chromatographic separation, and then concentrated and dried to produce a α-glucan dietary fiber product with target molecular weight 500-5000D.

2. The method of claim 1, wherein the Leuconostoc mesenteroides comprise one or more of the following strains: Leuconostoc mesenteroides CICC-23614, Leuconostoc mesenteroides LM-1226, Leuconostoc mesenteroides LM-0326, and Leuconostoc mesenteroides LM-31208.

3. The method of claim 1, wherein:
a slant seed medium for activation of the Leuconostoc mesenteroides comprises 15 g sucrose, 0.17 g peptone, 0.15 g Na₂HPO₄, 2 g agar, and added water to 100 mL;
a liquid seed activation medium comprises 10 g sucrose, 0.17 g peptone, and 0.15 g Na₂HPO₄, and added water to 100 mL;
a fermentation medium according to the liquid seed activation medium, or comprising a carbon source providing a sucrose concentration of 10-30%;
the nitrogen source comprises tryptone and yeast powder mixture; and
a pH is adjusted to between 6.8-7.0 and the slant seed medium, liquid seed activation medium, and fermentation medium is sterilized at 121° C. for 20 min.

4. The method of claim 1, wherein:
a lyophilized bacteria or the Leuconostoc mesenteroides strains preserved in liquid paraffin is streaked on a slant seed medium, and incubated at 25° C. for 24-48 h;
for a freeze-dried strain, a bacterial powder containing the Leuconostoc mesenteroides is dissolved with sterile water and inoculated 2 or 3 times on the slant seed medium;
2-3 loops of the Leuconostoc mesenteroides are inoculated by an inoculation loop in a 250 ml flask containing 100 ml of seed medium and then incubated at 100 rpm and 28° C. for 24 h to obtain a primary activated seed culture that is transferred to a 5 L automatic fermenter with 10% inoculum.

5. The method of claim 1, the carbon source comprises white sugar, sugarcane raw sugar, sucrose syrup, or combinations thereof, and the sucrose concentration is 10%, 15%, 18%, 20%, 25%, or 30%.

6. The method of claim 1, wherein the nitrogen source comprises tryptone, yeast powder, or mixture thereof.

7. The method of claim 1, wherein the nitrogen source comprises 0.3%.

8. The method of claim 1, wherein the temperature of the fermentation broth is 28° C.

9. The method of claim 1 wherein the dextranase comprises Dextranase Plus L

10. The method of claim 1, wherein the dextranase has an activity of 100 KDU/g.

11. The method of claim 1, wherein an amount of the dextranase is 1/10,000 of the total amount of fermentation culture.

12. The method of claim 1, wherein a total fermentation time is between 20-40 hours.

13. A method of preparing α-glucan, comprising the steps of:
in a fermentation vessel, inoculating an activated culture of Leuconostoc mesenteroides with inoculum and a fermentation broth comprising a carbon source that provides a sucrose concentration of between 10 and 30%, a nitrogen source of between 0.2% and 1.0%, and having a pH of between 6.8 and 7.0 at a temperature of between 25° C. and 28° C.; and
after between 5 and 30 hours of fermentation time, adding dextranase to the fermentation vessel, the dextranase comprising 1/10,0000 to 5/10,0000 by volume;
wherein the fermentation time and the amount of dextranase added is controlled to achieve a molecular weight of the α-glucan that is within 10000D.

14. The method of claim 13, wherein the Leuconostoc mesenteroides comprises Leuconostoc mesenteroides CICC-23614, Leuconostoc mesenteroides LM-1226, Leuconostoc mesenteroides LM-0326, Leuconostoc mesenteroides LM-31208, or combinations thereof.

15. The method of claim 13, wherein the Leuconostoc mesenteroides is activated in a slant seed medium comprising an aqueous mixture of between 10:1 and 15:1 sucrose, 0.17:1 peptone, 0.15:1 Na₂HPO₄, and 2:1 agar.

16. The method of claim 13, wherein the nitrogen source comprises tryptone, yeast powder, or combinations thereof.

17. The method of claim 13, wherein the inoculum comprises 10% by volume.

18. The method of claim 13, the carbon source comprises white sugar, sugarcane raw sugar, sucrose syrup, or combinations thereof.

19. The method of claim 1, wherein the nitrogen source comprises 0.3%.

20. The method of claim 1, wherein the temperature of the fermentation broth is 28° C.

21. The method of claim 1 wherein the dextranase comprises Dextranase Plus L having an activity of 100 KDU/g and is 1/10,000 of the total amount of fermentation culture.

22. The method of claim 1, wherein a total fermentation time is between 20-40 hours.

23. The method of claim 1, further comprising the steps of terminating the fermentation and drying the α-glucan to prepare dietary fiber products with a molecular weight of 500-5000D.
